(19) United States
(12) Patent Application Publication
(10) Pub. No.: US 2024/0299880 A1
(43) Pub. Date: Sep. 12, 2024

(54) METHOD FOR CAPTURING AND REMOVING CARBON DIOXIDE USING PHOTO-CULTIVATION

(71) Applicant: C&S INT Ltd., Gyeonggi-do (KR)

(72) Inventors: JooDong Son, Gyeonggi-do (KR); SooYoul Hong, Seoul (KR); KilYoung Lee, Gyeonggi-do (KR)

(21) Appl. No.: 18/566,473

(22) PCT Filed: Jun. 3, 2022
(86) PCT No.: PCT/KR2022/007947

§ 371 Date: Dec. 1, 2023

(30) Foreign Application Priority Data
Jun. 4, 2021 (KR) 10-2021-0072596

(51) Int. Cl. B01D 53/84; B01D 53/22; B01D 53/62; C12N 1/12; C12N 9/88

(52) U.S. Cl. CPC B01D 53/84; B01D 53/229; B01D 53/62; C12N 1/12; C12N 9/88; C12Y 402/01001; B01D 2255/804; B01D 2257/504; B01D 2258/0266; B01D 2258/06

(57) ABSTRACT

The present disclosure discloses a method for capturing and removing carbon oxide using photo-cultivation, the method including a carbonic anhydrase providing step in which carbonic anhydrase is provided to culture medium as a reaction catalyst; a carbon dioxide supplying step in which a processing object gas containing carbon dioxide is supplied to the carbonic anhydrase and the culture medium; a carbon dioxide converting step in which the carbon dioxide comes into contact with the carbonic anhydrase and the culture medium to convert the carbon dioxide into inorganic carbonic acid; and a carbon oxide consuming step in which the inorganic carbonic acid is employed and consumed in photosynthesis of microalgae contained in the culture medium.

### TECHNICAL FIELD

The present disclosure relates to a method of capturing and removing carbon dioxide from a gas containing carbon dioxide using photo-cultivation.

### BACKGROUND ART

Carbon dioxide may be produced in a variety of natural phenomenon, as well as industrial processes in which coal, oil and natural gas, and fuels are burned. Carbon dioxide is a substance that causes climate change or global warming, and as the importance of the environment is increased, regulations on its concentration in the atmosphere are being intensified. Therefore, various efforts are being made to reduce carbon dioxide emission or develop technologies for capturing carbon dioxide in an atmosphere. In addition, development for various technologies to utilize carbon dioxide in the atmosphere as a useful resource in biological processes is being underway. As a method for utilizing carbon dioxide, a method for using carbon dioxide directly and a method for converting carbon dioxide into other substance are being considered. However, since carbon dioxide is oxidized and is in a stable state, there is an aspect in which it is difficult to utilize carbon dioxide carbon dioxide directly in biological processes, and the like.

Meanwhile, a general photo-cultivation system may supply sunlight as an energy source and carbon dioxide as a carbon source to a culture medium containing microalgae. Therefore, the photo-cultivation system may function to consume carbon dioxide in the atmosphere, thereby reducing air pollution. However, the photo-cultivation system is mainly supplied with air containing carbon dioxide, and as mentioned above, carbon dioxide in the air is in a stable state, so there is a limitation in efficiently separating carbon dioxide from the atmosphere and using it.

### DISCLOSURE OF THE INVENTION

### Technical Problem

An object of the present disclosure is to provide a method for capturing and removing carbon dioxide, which efficiently captures and removes carbon dioxide from the processing object gas, using photo-cultivation of microalgae.

### Technical Solution

A method for capturing and removing carbon oxide according to one embodiment of the present disclosure includes a carbonic anhydrase providing step in which carbonic anhydrase is provided to culture medium as a reaction catalyst; a carbon dioxide supplying step in which a processing object gas containing carbon dioxide is supplied to the carbonic anhydrase and the culture medium; a carbon dioxide converting step in which the carbon dioxide comes into contact with the carbonic anhydrase and the culture medium to convert the carbon dioxide into inorganic carbonic acid; and a carbon oxide consuming step in which the inorganic carbonic acid is employed and consumed in photosynthesis of microalgae contained in the culture medium.

Also, the method for capturing and removing carbon oxide of the present disclosure may further include a filtering step in which the processing object gas is filtrated to increase a carbon oxide concentration of the processing object gas.

In addition, in the filtering step, the carbon dioxide concentration of the processing object gas may be increased by 10 times or more compared to a carbon dioxide concentration in air.

Further, the carbonic anhydrase may be enzyme derived from Gracilariopsis chorda or Sulfurihydrogenibium azorense. Also, a gene sequence of the carbonic anhydrase derived from Gracilariopsis chorda may include a nucleotide sequence encoding an amino acid sequence of SEQ ID NO. 1, and a gene sequence of the carbonic anhydrase derived from Sulfurihydrogenibium azorense may include a nucleotide sequence encoding an amino acid sequence of SEQ ID NO. 11.

In the carbonic anhydrase providing step, in addition, the carbonic anhydrase may be provided in a state of being fixed to a catalyst fixture. Further, the catalyst fixture may be a natural scrubber.

Also, the carbon dioxide consuming step may be performed while additionally supplying separate air along with sunlight to the culture medium.

### Advantageous Effects

The method for capturing and removing carbon oxide of the present disclosure may efficiently capture and remove carbon dioxide since carbon dioxide is converted into inorganic carbonic acid using water and carbonic anhydrase acting as a reaction catalyst, and is then used in a photo-cultivation of microalgae.

Also, the method for capturing and removing carbon oxide may efficiently capture and remove carbon dioxide contained in a processing object gas, such as air containing carbon dioxide, a gas discharged from livestock farms, manufacturing plants or the like.

In addition, the method for capturing and removing carbon oxide may increase carbon dioxide capture efficiency since carbon dioxide, which is directly separated from air and concentrated, comes into contact with carbonic anhydrase acting as a reaction catalyst.

Further, the method for capturing and removing carbon oxide may reduce carbon dioxide in air more efficiently because it separates and concentrates carbon dioxide in air using a membrane filter, and then uses it.

Also, the method for capturing and removing carbon oxide of the present disclosure consumes and removes carbon dioxide, which is converted into inorganic carbonic acid, in a photo-cultivation of microalgae, so there is no need to additionally require a separate device configured to decompose captured carbon dioxide, thereby reducing a cost.

In addition, the method of capturing and removing carbon dioxide captures carbon dioxide and supplies it in the photo-cultivation of microalgae, so it is possible to reduce a cost for the photo-cultivation of microalgae.

### BRIEF DESCRIPTION OF THE DRAWINGS

FIG. 1 is a process flow chart describing the method for capturing and removing carbon oxide using photo-cultivation, according to one embodiment of the present disclosure.

### BEST MODE FOR CARRYING OUT THE INVENTION

Hereinafter, a method for capturing and removing carbon dioxide using photo-cultivation, according to one embodiment of the present disclosure is described.

First, a method for capturing and removing carbon dioxide using photo-cultivation, according to one embodiment of the present disclosure is described.

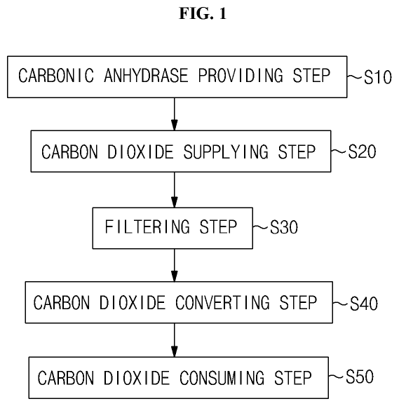

FIG. 1 is a process flow chart describing the method for capturing and removing carbon oxide using photo-cultivation, according to one embodiment of the present disclosure.

Referring to FIG. 1, the method for capturing and removing carbon oxide using photo-cultivation, according to one embodiment of the present disclosure may include a carbonic anhydrase providing step S10, a carbon dioxide supplying step S20, a carbon dioxide converting step S40, and a carbon oxide consuming step S50. The method for capturing and removing carbon oxide may further include a filtering step S30.

The method for capturing and removing carbon oxide may convert carbon dioxide into inorganic carbonic acid and capture it using carbonic anhydrase as a reaction catalyst, thereby increasing carbon dioxide capture efficiency.

In addition, the method for capturing and removing carbon oxide may filter selectively carbon dioxide from the processing object gas, using a membrane filter and bring it into contact with carbonic anhydrase, thereby increase carbon dioxide capturing efficiency. Here, the processing object gas may refer to air or exhaust gas discharged from livestock farms, plants or the like and containing carbon dioxide. The exhaust gas may be a gas having a carbon dioxide concentration higher than that of air.

Additionally, the method for capturing and removing carbon oxide may dissolve carbon dioxide in water in the form of inorganic carbonic acid and capture it, thereby increasing carbon dioxide capturing efficiency.

The method for capturing and removing carbon oxide of the present disclosure consumes and removes carbon dioxide, which is converted into inorganic carbonic acid, in a photo-cultivation of microalgae, so there is no need to additionally require a separate device configured to decompose captured carbon dioxide.

In addition, the method of capturing and removing carbon dioxide captures carbon dioxide and supplies it in the photo-cultivation of microalgae, so it may be applied to the photo-cultivation process of microalgae.

The carbonic anhydrase providing step S10 is the step of providing carbonic anhydrase to culture medium as a reaction catalyst. Carbonic anhydrase may be provided to the culture medium of microalgae. Carbonic anhydrase may act as a reaction catalyst by which water and carbon dioxide are reacted with each other, to produce inorganic carbonic acid. Here, the above water may be water in the culture medium. Carbonic anhydrase may react carbon dioxide and water to each other to produce inorganic carbonic acid (HCO₃₋) according to the chemical formula 1 below.

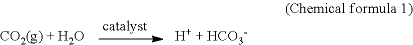

The carbonic anhydrase may act as a reaction catalyst that converts carbon dioxide into inorganic carbonic acid. The carbonic anhydrase (CA) is a metal enzyme containing zinc therein and may refer to enzyme that promotes the hydration reaction of carbon dioxide. The carbonic anhydrase may be an enzyme derived from Gracilariopsis chorda or Sulfurihydrogenibium azorense. Also, a gene sequence of carbonic anhydrase derived from Gracilariopsis chorda may include a nucleotide sequence encoding an amino acid sequence of SEQ ID NO. 1. In addition, a gene sequence of carbonic anhydrase derived from Gracilariopsis chorda may include a nucleotide sequence of SEQ ID NO. 2. Furthermore, a gene sequence of carbonic anhydrase derived from Sulfurihydrogenibium azorense may include a nucleotide sequence encoding an amino acid sequence of SEQ ID NO. 11. Also, a gene sequence of carbonic anhydrase derived from Sulfurihydrogenibium azorense may include a nucleotide sequence of SEQ ID NO. 12. The carbonic anhydrase may be the α-type derived from Neisseria gonorrhoeae. The carbonic anhydrase may be carbonic anhydrase derived from Hydrogenovibrio marinus. The carbonic anhydrase may be the α, β, γ, δ, and ε type carbonic anhydrases that exist in nature. In addition, various previously known enzymes may be used as the carbonic anhydrase.

The carbonic anhydrase may be provided in a state of being fixed to a catalyst fixture. The catalyst fixture may be a natural scrubber, mesh net, or porous material made of cellulose material extracted from natural pulp. Since the catalyst fixture is made of cellulose material, it can effectively fix carbonic anhydrase to carbohydrate. The catalyst fixture may fix carbonic anhydrase in pores existing on a surface and the inside thereof. The catalyst fixture may fix carbonic anhydrase at 3 to 10 mg/2,000 cm².

The catalyst fixture can be placed and immersed inside the culture medium in a state where carbonic anhydrase is fixed on a surface thereof. Therefore, the catalyst fixture may allow carbonic anhydrase to come into contact with water.

The carbon dioxide supplying step S20 is the step of supplying processing object gas containing carbon dioxide to bring it into contact with water and carbonic anhydrase. As mentioned above, the processing object gas may be air, or a gas discharged from livestock farms, plants or the like and containing carbon dioxide having a concentration higher than that of carbon dioxide in air. The water may be a culture medium. In the carbon dioxide supplying step S20, the processing object gas may be suctioned from the outside and then supplied. Among the gases to be treated, air may have a carbon dioxide concentration of 400 to 420 ppm. Additionally, the air may further contain other components such as nitrogen. In addition, among the gases to be treated, a gas discharged from livestock farms, plants or the like may contain carbon dioxide having a concentration higher than that of carbon dioxide in the air. In the carbon dioxide supplying step S20, air in the atmosphere may be suctioned and supplied using a separate vacuum pump or a suction pump.

The filtering step S30 is the step of filtering a processing object gas to increase a carbon dioxide concentration of a processing object gas. The filtering step S30 may be optionally performed when a carbon dioxide concentration of processing object gas is low. For example, the filtering step S30 may be performed when a processing object gas is air. The filtering step S30 may be performed before supplying a processing object gas to carbonic anhydrase. For example, the filtering step S30 may be performed after the carbon dioxide supplying step S20. In the filtering step S30, a processing object gas having an increased carbon dioxide concentration may be supplied. For example, as described above, other gases such as nitrogen or the like and carbon dioxide are mixed in air, so a carbon dioxide concentration of air is relatively low. Therefore, in the filtering step S30, air is filtered to enable a gas (to be treated) having a carbon dioxide concentration higher than that of air to be supplied to carbonic anhydrase. In the filtering step S30, a carbon dioxide concentration of processing object gas may be increased by 10 times or more compared to a carbon dioxide concentration in air. In the filtering step S30, a carbon dioxide concentration of processing object gas may be preferably increased 10 to 30 times. In the filtering step S30, a processing object gas having a carbon dioxide concentration of 5,000 ppm or more may be preferably produced. In addition, in the filtering step S30, a processing object gas having a carbon dioxide concentration of 10,000 ppm or more may be preferably produced. Therefore, the processing object gas may increase a dissolved carbon dioxide concentration dissolved in the culture medium.

In the filtering step S30, a separation membrane module that selectively separates carbon dioxide may be utilized. The separation membrane module may include a separation membrane and an absorbent. The separation membrane may separate air and the absorbent and increase an effective contact area between air and the absorbent, required to transfer carbon dioxide from air to the absorbent. The separation membrane may be formed from a porous thin film having hydrophobicity. The separation membrane may be made of polytetrafluoroethylene (PTFE) material, polydimethylsiloxane (PDMS) material, polyetherether ketone (PEEK) material, or polyvinylidene fluoride (PVDF) material. The absorbent material may be distilled water. Additionally, the absorbent may be NaOH or KOH.

The carbon dioxide converting step S40 is the step of converting carbon dioxide into inorganic carbonic acid by bringing it into contact with water and carbonic anhydrase. The carbon dioxide may be carbon dioxide contained in a processing object gas. Carbon dioxide contained in the processing object gas may come into contact with water, and be converted into inorganic carbonic acid through a catalytic action of carbonic anhydrase. The inorganic carbonic acid may be bicarbonate (HCO₃⁻). Here, the water may be water of culture medium.

The carbon dioxide converting step S40 may be performed while additionally supplying separate air to culture medium. The air may give an aeration effect to culture medium. The air may be supplied to culture medium in the form of bubbles. Therefore, the processing object gas may come into contact with carbonic anhydrase more efficiently. Additionally, in the carbon dioxide converting step S40, carbon dioxide of 0.04% contained in air that is additionally supplied air may also be converted.

The carbon dioxide consuming step S50 is the step in which inorganic carbonic acid is employed and consumed in photosynthesis of microalgae contained in culture medium. The culture medium may contain microalgae cultured by photosynthesis. The inorganic carbonic acid may be consumed while being used in photosynthesis of microalgae. In the carbon dioxide consuming step S50, by allowing inorganic carbonic acid, which is formed, to come into direct contact with microalgae, a conversion of inorganic carbonic acid back to carbon dioxide can be minimized. That is, the culture medium can be formed so that water, carbonic anhydrase and microalgae come into contact with each other. Therefore, the carbon dioxide consuming step S50 may be performed almost simultaneously with the carbon dioxide converting step S40. The inorganic carbonic acid is used by microalgae immediately after being converted from carbon dioxide, so it may not be converted into carbon dioxide. The culture medium may be maintained at a temperature of 15 to 70° C. The carbonic anhydrase may be maintained in a temperature range which is suitable for microalgae to be active.

In the carbon dioxide consuming step S50, by allowing carbon dioxide converted in the form of inorganic carbonic acid to be used for photosynthesis of microalgae, carbon dioxide can be removed from a processing object gas. The microalgae may fix carbon dioxide as carbohydrate while using inorganic carbonic acid as an energy source during photosynthesis.

The carbon dioxide consuming step S50 may be performed while additionally supplying separate air along with sunlight to culture medium. The air may give an aeration effect to culture medium. The air may be supplied to culture medium in the form of bubbles. Therefore, the treated inorganic carbonic acid may efficiently come into contact with microalgae.

Meanwhile, the method for capturing and removing carbon oxide may, among carbon dioxide supplied to culture medium, capture and filtrate carbon dioxide that is not consumed as an energy source and discharged, again and then supply it to culture medium again. Therefore, the method for capturing and removing carbon oxide may not emit carbon dioxide to the outside.

The following describes specific embodiments of the method for capturing and removing carbon dioxide, according to the present disclosure.

### 1. Evaluation of Dissolved Carbon Dioxide Concentration in Culture Medium

Table 1 shows the measurement results of dissolved carbon dioxide concentration of culture medium. In this evaluation, a dissolved carbon dioxide concentration of culture medium over supplying time was measured while carbonic acid solution using carbon dioxide source gas having a carbon dioxide concentration of 10,000 ppm obtained by filtering air was supplied to culture medium. In addition, in this evaluation, a dissolved carbon dioxide concentration was measured in the same manner while carbonic acid solution using unfiltered air as a carbon dioxide source gas was supplied to culture medium. The carbonic acid solution was supplied with air to allow it to be aerated. The dissolved carbon dioxide concentration of culture medium was measured after 10 minutes, 30 minutes, and 60 minutes, respectively.

TABLE 1
Measurement results of dissolved carbon
dioxide concentration of culture medium.

| | | Supplying time (Minutes) | | | |
|---|---|---|---|---|---|
| | | 0 | 10 | 30 | 60 |
| Condition | | minutes | minutes | minutes | minutes |
| Dissolved | Filtered air supply condition | 375 | 1,150 | 1,400 | 1,420 |
| carbon dioxide | (Carbon dioxide | | | | |
| concentration | concentration: 10,000 ppm) | | | | |
| (ppm) | Unfiltered air supply | 375 | 450 | 675 | 978 |
| | condition | | | | |

As shown in Table 1, it can be confirmed that when air is supplied to culture medium as a processing object gas, the dissolved carbon dioxide concentration of culture medium is increased. In addition, when air is filtered to have the carbon dioxide concentration of 10,000 ppm and supplied, the dissolved carbon dioxide concentration in culture medium is measured to be higher than that when unfiltered air is supplied. Therefore, it can be confirmed that carbon dioxide in the air can be captured and removed by the carbon dioxide capture and removal method of the present disclosure. In addition, it can be confirmed that the dissolved carbon dioxide concentration of culture medium can be effectively increased utilizing carbon dioxide in the air by the method for capturing and removing carbon oxide of the present disclosure.

As shown in Table 1, it can be confirmed that the dissolved carbon dioxide concentration is increased as the supply time is elapsed when carbon dioxide is supplied to culture medium by the method for capturing and removing carbon oxide of the present disclosure.

### 2. Microalgae Cultivation Test for Culture Medium

Table 2 shows microalgae cultivation test results for culture medium. In this evaluation, the degree of cultivation of microalgae was evaluated while carbonic acid solution according to the method for capturing and removing carbon oxide of the present disclosure was supplied to culture medium. A mesh net woven using natural scrubber was used as the catalyst fixture that fixes carbonic anhydrase. Carbonic anhydrase was fixed to a surface of the catalyst fixture at approximately 4 mg/2,000 cm². In addition, air was used as a processing object gas, and was filtered to have a carbon dioxide concentration of 10,000 ppm. The number of microalgae cells in culture medium was evaluated by measuring an absorbance of a spectrometer. The number of microalgae cells was measured after 8 hours, 16 hours, 20 hours, and 24 hours, respectively.

TABLE 2
Microalgae cultivation test results for culture medium.

| | | Cultivation time (Hours) | | | | |
|---|---|---|---|---|---|---|
| Condition | | 0 | 8 | 16 | 20 | 24 |
| Absorbance | Using 10,000 ppm | 0.615 | 1.347 | 1.857 | 2.121 | 2.210 |
| | carbon oxide/carbonic | | | | | |
| | anhydrase | | | | | |
| | Without using 10,000 | 0.613 | 0.943 | 1.393 | 1.591 | 1.724 |
| | ppm carbon oxide/ | | | | | |
| | carbonic anhydrase | | | | | |

As shown in Table 2, it can be confirmed that when a processing object gas, which contains carbon dioxide having a high concentration obtained by filtering air, and carbonic anhydrase are utilized, the culture growth of microalgae can be effectively performed. In view of the absorbance measurement results of culture medium, it can be confirmed that the absorbance is increased over time and the photo-cultivation of microalgae is relatively largely increased. Therefore, it can be confirmed that the amount of carbon dioxide used for photosynthesis of microalgae is increased among supplied carbon dioxide and the amount of removed carbon dioxide is increased. In comparison, when carbonic anhydrase is not used, the absorbance of culture medium is relatively low. Therefore, it can be confirmed that the photo-culture of microalgae is carried out relatively low and the amount of carbon dioxide used for photosynthesis of microalgae is small among supplied carbon dioxide.

So far, the present disclosure has been examined in detail, focusing on preferred embodiments. These embodiments are not intended to limit the present disclosure, but are merely illustrative, and should be considered from a descriptive rather than a definitive point of view. The true technical protection scope of the present disclosure should be determined by the technical spirit of the appended claims, not by the foregoing description.

1. A method for capturing and removing carbon oxide, comprising:
a carbonic anhydrase providing step in which carbonic anhydrase is provided to culture medium as a reaction catalyst;
a carbon dioxide supplying step in which a processing object gas containing carbon dioxide is supplied to the carbonic anhydrase and the culture medium;
a carbon dioxide converting step in which the carbon dioxide comes into contact with the carbonic anhydrase and the culture medium to convert the carbon dioxide into inorganic carbonic acid; and
a carbon oxide consuming step in which the inorganic carbonic acid is employed and consumed in photosynthesis of microalgae contained in the culture medium.

2. The method for capturing and removing carbon dioxide of claim 1, further comprising a filtering step in which the processing object gas is filtrated to increase a carbon oxide concentration of the processing object gas.

3. The method for capturing and removing carbon dioxide of claim 2, wherein, in the filtering step, the carbon dioxide concentration of the processing object gas is increased by 10 times or more compared to a carbon dioxide concentration in air.

4. The method for capturing and removing carbon dioxide of claim 1, wherein the carbonic anhydrase is enzyme derived from Gracilariopsis chorda or Sulfurihydrogenibium azorense.

5. The method for capturing and removing carbon dioxide of claim 4, wherein a gene sequence of the carbonic anhydrase derived from Gracilariopsis chorda includes a nucleotide sequence encoding an amino acid sequence of SEQ ID NO. 1, and a gene sequence of the carbonic anhydrase derived from Sulfurihydrogenibium azorense includes a nucleotide sequence encoding an amino acid sequence of SEQ ID NO. 11.

6. The method for capturing and removing carbon dioxide of claim 1, wherein, in the carbonic anhydrase providing step, the carbonic anhydrase is provided in a state of being fixed to a catalyst fixture.

7. The method for capturing and removing carbon dioxide of claim 6, wherein the catalyst fixture is a natural scrubber.

8. The method for capturing and removing carbon dioxide of claim 1, wherein the carbon dioxide consuming step is performed while additionally supplying separate air along with sunlight to the culture medium.
